(19) United States
(12) Patent Application Publication
(10) Pub. No.: US 2021/0402200 A1
(43) Pub. Date: Dec. 30, 2021

(54) METHOD AND APPARATUS FOR MODULATION OF TRACTS IN NERVOUS TISSUE

(71) Applicant: WEINBERG MEDICAL PHYSICS INC., North Bethesda, MD (US)

(72) Inventor: Irving WEINBERG, North Bethesda, MD (US)

(21) Appl. No.: 17/358,696

(22) Filed: Jun. 25, 2021

Related U.S. Application Data
(60) Provisional application No. 63/044,111, filed on Jun. 25, 2020.

(51) Int. Cl. A61N 2/00; A61N 2/02; A61N 2/06

(52) U.S. Cl. CPC A61N 2/006; A61B 5/05; A61N 2/06; A61N 2/02

(57) ABSTRACT

An apparatus and method for generating magnetic field changes that more accurately conform to the tracts and regions of the brain that includes at least two coils for generating magnetic fields. Timing and/or phase of the magnetic fields generated by the at least two coils establishes relative perceived motion and/or directionality within the nervous tissue

### CROSS REFERENCE AND PRIORITY CLAIM

This application claims priority to U.S. Provisional Patent Application Ser. No. 63/044,111, entitled “METHOD AND APPARATUS FOR MODULATION OF TRACTS IN NERVOUS TISSUE” filed Jun. 25, 2020, the entirety of which is incorporated by reference.

### FIELD

Disclosed embodiments are directed, generally, to a method and apparatus for modulating tracts in nervous tissue.

### BACKGROUND

It is known that the activity of selected regions in the brain may be modulated through the application of dynamically changing magnetic fields. The clinical application of this phenomenon is known as “transcranial magnetic stimulation”. The spatial extent of the region in which magnetic fields are changing is determined by the shape of the electrical coils located in the vicinity of the brain, and the amount of current flowing through those coils. The shape of the regions affected by the dynamic magnetic fields is generally limited by coil design considerations to oval regions that are centered near the skull. Many of the brain tracts that are most useful for research or clinical applications are not of these shapes and locations. For example, emotional disturbances are believed to arise from deep-seated regions in the brain, and the neuronal transmissions associated with such disturbances may project in linear fashion. The term modulation is meant to include stimulation and inhibition or interference with transmission of neuronal information or control.

It is known that information about such dynamically changing magnetic fields may be detected through the alteration of magnetic resonance images, as taught by D. E. Bohning et al in the 1977 Neuroreport publication entitled “Magnetic Transcranial Magnetic field Stimulation (TMS) fields in vivo with Magnetic Resonance Imaging (MRI). In that publication, a coil was inserted within a superconducting MRI, and a current passed through the coil in order to create dynamically changing magnetic fields. The use of such system can be dangerous to a subject, since there may be a force or torque on said coil during the current passage through the coil, said force or torque being generated by the interaction of the static magnetic field of the superconducting magnet and the transient magnetic field of the current-carrying coil. It is known that TMS devices are supplied with specific coil configurations optimized for activating specific regions of nervous tissues, and that in some cases the coils are physically moved in order to activate those regions. It is known that the amount of current sent to said TMS coil may be prescribed on the basis of observed physiological effects such as finger twitching.

### SUMMARY

Disclosed embodiments describe an apparatus and method for generating magnetic field changes that more accurately conform to the tracts and regions of the brain that are of interest to researchers and clinicians.

In some embodiments, an apparatus comprises at least two coils configured to be arranged in the vicinity of nervous tissue and to generate magnetic fields for modulation of the nervous tissue, and a control system configured to control timing of magnetic fields applied for modulation of the nervous tissue. The timing may be within the integration time of the nervous tissue.

In some embodiments, the timing and/or phase of the magnetic fields generated by the at least two coils establishes relative perceived motion and/or directionality within the nervous tissue.

### BRIEF DESCRIPTION OF FIGURES:

FIG. 1 illustrates an example of a human or other animal at various times in the vicinity of at least a coil, electromagnet, or electropermanent magnet;

FIG. 2 is a block diagram of a control system and current source according to the disclosed embodiments; and

FIG. 3 is a flow chart of a method of modulating nervous tissue according to the disclosed embodiments.

### DETAILED DESCRIPTION

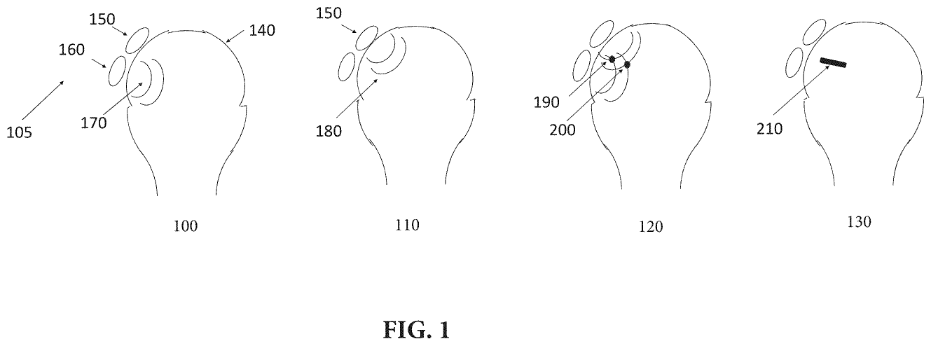

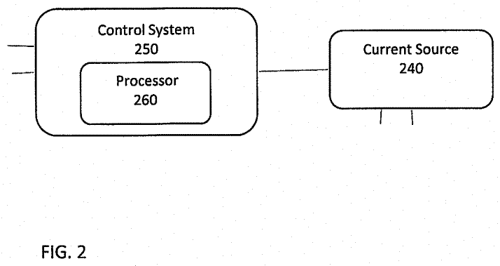

FIG. 1 shows an example of one embodiment of invention apparatus 105. The head of a human or other animal 140 is shown at various times 100, 110, 120, 130. At least one coil or electromagnet or electropermanent magnet 150 is in the vicinity (i.e., within one meter) of the head 140. At least one additional such coil or electromagnet or electropermanent magnet 160 is similarly located in the vicinity (i.e., within one meter) of the head 140. At time corresponding to 100, coil 150 is activated by a current from a system to generate magnetic field 170 in head 140. The source of the current 240 and the control system 250, including a processor 260, are illustrated in FIG. 2, which can connect via wires or wirelessly to the apparatus 105. At a subsequent time that is shorter than a neuronal response time for example, less than 0.1 seconds, less than 100 milliseconds, less than 500 microseconds as described in U.S. Pat. No. 9,411,030 incorporated by reference, the system generates a current through coil 160 to generate magnetic field 180 in head, as shown in 110. Time 120 represents the magnetic field as perceived by the brain, in which case the physiological effects of the magnetic fields of times 100 and 110 are integrated or otherwise combined at locations 190 and 200. As shown in time 130, the summed effects may activate or otherwise modulate a tract 210 in the brain.

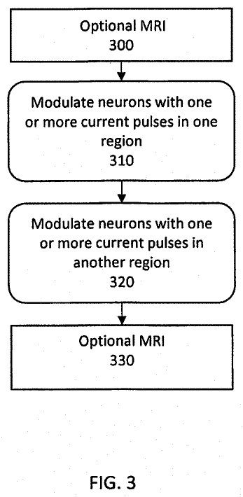

The above operations in one example of a method are illustrated in the flow chart of FIG. 3. Although the term “subsequent operation” is used in the next section of this specification for illustration of the method of the invention, It should be understood that some Operations may be in different orders and may be repeated. The sequence may start at 300 with an MRI of the brain or other nervous organ (e.g., spinal cord). A current pulse is sent through a coil or electromagnet or electropermanent magnet 310. Another current pulse is sent through a coil or electromagnet or electropermanent magnet 320. The pulse at 320 might also be from the same coil as at 310. Additional similar operations at 320 may be applied. The brain and/or nervous tissues may integrate operations 310 and 320.

Optionally, a magnetic resonance (MR) image may be obtained to determine the magnetic fields generated in any or all of the above operations. This determination may include examination of effect of the magnetic fields on the spin states at various locations and times, for example as taught by D. E. Bohning et al (cited above).

As discussed above in the description of the Figures, the apparatus 105 comprise at least two coils or electromagnets or electropermanent magnets 150 and 160 within one meter of neuronal or nervous tissue such as brain 140. It should be understood that the terms “neuronal” or “nervous” tissue refers to tissues containing nerves or neurons. In FIG. 1, the nervous tissue is represented as a brain-containing head 140.

It should be understood that the term “electropermanent magnet” includes an apparatus including at least one coil or current-carrying material (for example, a wire) and magnetizable material, wherein the magnetization of the magnetizable material changes in magnitude or direction as a result of the current, and at least some of this change in magnetization persists after a current is run through the at least one coil or current-carrying material. For the purposes of this disclosure, the term “coil” includes coils, portions of current-carrying material, electromagnets and electropermanent magnets. For the purposes of this disclosure, the phrase “at least two coils configured to be arranged in the vicinity of nervous tissue and to generate magnetic fields for modulation of the nervous tissue” is meant to include generation of magnetic fields by the coil, the the magnetizable material, or the combination of both the coil and the magnetizable material.

In accordance with disclosed embodiments, the coils 150 and 160 and other possible coils may be activated with electrical currents to create magnetic fields in the subject, e.g., the subject's head. The electrical currents to the various coils may be generated at sequential times so that the neuronal tissue perceives a cumulative magnetic effect with a region that is larger or different than the region of any one coil. The direction of the magnetic effect may be controlled by changing the time, phase, and magnitude of currents through the coils. It should be understood that the term “magnetic effect” may include the induction of electric fields that may stimulate, inhibit, or otherwise modulate physiological activities of nervous tissue. The physiological effect of the magnetic effect on nervous tissue is denoted “physiological effect”.

For the purposes of this disclosure the terms “integrate” and “integration” are meant to describe an operation wherein the response time of the nervous tissue (e.g., the integration time) to successive magnetic effects is less than the time between the start and/or end of each magnetic effect, or is less than the time between the start of end of all magnetic effects. The integration time may include the time for a single neuron or nerve to respond to modulation, the time for a group or neurons or nerves to respond to modulation, or the time for a circuit containing a group of neurons or nerves to respond to modulation.

For the purposes of this disclosure, the term “integration time” is meant to be comparable in length with neuronal response times (as described above). Neuronal response times may be several milliseconds, or tens or milliseconds, hundreds of milliseconds, or longer, depending on the type of neuron or nerve and the number of neurons or nerves involved in the response.

The terms “integrate” and “integration” also are meant to include partial overlap or minimal separation (for example less than 1 second) of the magnetic effects in time, to establish a perceived relative motion and/or direction of successive magnetic effects at various locations. As an example, if a magnetic effect is applied to a subject's brain by a coil (for example, coil 150 at the location of coil 150) and then another magnetic effect is applied by another coil (for example, coil 160 at the location of coil 160), and these magnetic effects overlapped within an integration time, then a circuit in the brain may be modulated (via physiological effect) as if both coils 150 and 160 were activated at the same time at their respective locations. It is understood that this process may be applied to create physiological effects with many sizes and shapes.

If the magnetic effects of the two or more coils were not applied at the same time, then the physiological effect may be directional within a neuronal pathway or tract. The difference in timing is denoted as “phase” in this disclosure. Adjustments in the phase of the magnetic fields generated by the coils may be used to further specify the size, magnitude, location, or direction of such modulation. It should be understood that the use of MR or magnetic particle imaging may assist in making such adjustments, for example through a feedback routine that examines the magnetic fields through MR or magnetic particle imaging and then changes the magnetic fields.

Operations in the method are shown in FIG. 3. One or more imaging studies 300 and 330 may be obtained prior to, during, and/or after activation of the coils, in order to add information concerning the spatial extent or other description of the applied magnetic fields and of the response of the nervous tissue (for example, with the BOLD effect). The imaging study may be obtained with magnetic resonance imaging, electron resonance imaging, magnetic particle imaging, or other means that employ the electropermanent magnets used to perform the modulation. It is understood that the electropermanent magnets may be used to collect images, so that TMS and imaging may be performed without moving the subject from one platform to another. An advantage of the invention is that without such motion, position accuracy is better and the timing between TMS and MRI is reduced so that the measurement of the response of nervous tissue is more accurate. The use of electropermanent magnets to collect images was previously described in issued patent U.S. Pat. No. 10,908,240 and in related patents, incorporated herein by reference.

It should be understood that the use of electropermanent magnets in a single apparatus to generate the magnetic fields for modulation of nervous tissue and to demonstrate anatomy and physiological responses of the nervous tissue and to describe the extent of the magnetic fields generated to modulate the nervous tissue is novel. Since the one or more coils or electropermanent magnets may be turned on and off or otherwise modulated so that there is no static magnetic field being applied at the time that the TMS pulse is applied by one or more coils or electropermanent magnets, there will be no force on the one or more coils. Without the attendant motion that may accompany forces, such an apparatus may be safer to use than the system described by Bohning et al. Thus, the lack of a permanent static field provides additional safety and flexibility to the user of the apparatus.

It should be understood that the combinatory use of multiple electropermanent magnets with appropriate phasing as described herein may be more effective in modulating nervous tissues that are deep in the brain as compared to the one to four coils presently used in TMS.

It is understood that the timing and activation magnitudes of multiple coils may represent an advantage in flexibility over TMS devices that have specific configurations optimized to modulate specific regions. As an example, a user may choose to use the apparatus to modulate one region in a subject, and then modulate a different region in the same or another subject, without having to physically move the coil locations, but only having to change the electrical parameters (for example, the phase and/or magnitude) of the currents supplied to the at least two coils in the invention. The invention therefore corresponds to an “all-purpose” system that might be used by a medical practitioner to treat many conditions.

It should be understood that the apparatus and method of the invention may be used in psychiatric disorders or illnesses to habituate a subject or otherwise modulate a desired tract or pathway. It should be understood that the apparatus and method of the invention may be used in neurological disorders or illnesses to habituate a subject or otherwise modulate a desired tract or pathway, for example prior to surgery or other ablative procedures.

Moreover, those skilled in the art will recognize, upon consideration of the above teachings, that the above exemplary embodiments and the control system may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out he above-described method operations and resulting functionality. In this case, the term “non-transitory” is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

While various exemplary embodiments have been described above, it should be understood that they have been presented by way of example only, and not limitation. Thus, the breadth and scope of the present invention should not be limited by any of the above-described exemplary embodiments, but should instead be defined only in accordance with the following claims and their equivalents.

For example, in accordance with some embodiments An apparatus comprises at least two coils configured to be arranged in the vicinity of nervous tissue and to generate magnetic fields for modulation of the nervous tissue, and a control system configured to control timing of magnetic fields applied for modulation of the nervous tissue wherein the timing is within the integration time of the nervous tissue.

The modulation created by the magnetic fields from the at least two coils in combination is different in size, shape, direction, or magnitude than the modulation that each coil creates separately.

The at least two coils are within electropermanent magnets.

The at least two coils are configured to be arranged within 1 meter of the nervous tissue.

The control system is configured to activate a second coil of the at least two coils subsequent to activation of a first coil of the at least two coils.

The first coil and the second coil are configured to generate overlapping magnetic fields in the nervous tissue.

The integration time is less than 1 second.

The integration time is several milliseconds, or tens or milliseconds, or hundreds of milliseconds, or longer.

The apparatus is configured to habituate a subject or otherwise modulate a desired tract or pathway.

The apparatus is, further configured, by selecting electrical parameters of the at least two coils, to modulate one ore more regions in one subject, and to modulate one or more different regions in the same or different subject or a different subject by adjusting the electrical parameters of the at least two coils.

The apparatus is configured to both collect data used to form an image of the nervous tissue in a subject and to modulate said nervous tissue, without moving the subject.

In accordance with at least some embodiments a method of modulating nervous tissue comprises: positioning at least two coils in the vicinity of nervous tissue, generating magnetic fields by activating the at least two coils, and applying the magnetic fields to modulate the nervous tissue, wherein timing of magnetic fields applied for modulation of said nervous tissue is within the integration time of the nervous tissue.

The magnetic fields from the at least two coils may be used to perform an imaging study before, after or during the modulation.

The timing and/or phase of the magnetic fields generated by the at least two coils establishes relative perceived motion and/or directionality within the nervous tissue.

The at least two coils are positioned within 1 meter of the nervous tissue.

A second coil of the at least two coils is activated subsequent to activation of a first coil of the at least two coils.

The first coil and the second coil generate overlapping magnetic fields in the nervous tissue.

The integration time is less than 1 second.

The integration time is several milliseconds, or tens or milliseconds, or hundreds of milliseconds, or longer.

The method further comprises habituating a subject or otherwise modulating a desired tract or pathway.

The method further comprises modulating one or more regions in one subject and then modulating one or more different regions in the same or different subject or a different subject.

The magnetic fields are applied to both collect data used to form an image of the nervous tissue in a subject and to modulate said nervous tissue, without moving the subject.

1. An apparatus comprising:
at least two coils configured to be arranged in the vicinity of nervous tissue and to generate magnetic fields for modulation of the nervous tissue, and
a control system configured to control timing of magnetic fields applied for modulation of the nervous tissue wherein the timing is within the integration time of the nervous tissue.

2. The apparatus of claim 1, wherein the modulation created by the magnetic fields from the at least two coils in combination is different in size, shape, direction, or magnitude than the modulation that each coil creates separately.

3. The apparatus of claim 1, wherein the at least two coils are within electropermanent magnets.

4. The apparatus of claim 3, wherein the at least two coils are configured to be arranged within 1 meter of the nervous tissue.

5. The apparatus of claim 1, wherein the control system is configured to activate a second coil of the at least two coils subsequent to activation of a first coil of the at least two coils.

6. The apparatus of claim 5, wherein the first coil and the second coil are configured to generate overlapping magnetic fields in the nervous tissue.

7. The apparatus of claim 1, wherein the integration time is less than 1 second.

8. The apparatus of claim 1, configured to habituate a subject or otherwise modulate a desired tract or pathway.

9. The apparatus of claim 1, further configured, by selecting electrical parameters of the at least two coils, to modulate one ore more regions in one subject, and to modulate one or more different regions in the same or different subject or a different subject by adjusting the electrical parameters of the at least two coils.

10. The apparatus of claim 1 configured to both collect data used to form an image of the nervous tissue in a subject and to modulate said nervous tissue, without moving the subject.

11. A method of modulating nervous tissue comprising:
positioning at least two coils in the vicinity of nervous tissue,
generating magnetic fields by activating the at least two coils, and
applying the magnetic fields to modulate the nervous tissue, wherein timing of magnetic fields applied for modulation of said nervous tissue is within the integration time of the nervous tissue.

12. The method of claim 11, where the magnetic fields from the at least two coils may be used to perform an imaging study before, after or during the modulation.

13. The method of claim 11, where the timing and/or phase of the magnetic fields generated by the at least two coils establishes relative perceived motion and/or directionality within the nervous tissue.

14. The method of claim 11, wherein the at least two coils are positioned within 1 meter of the nervous tissue.

15. The method of claim 11, wherein a second coil of the at least two coils is activated subsequent to activation of a first coil of the at least two coils.

16. The method of claim 15, wherein the first coil and the second coil generate overlapping magnetic fields in the nervous tissue.

17. The method of claim 11, wherein the integration time is less than 1 second.

18. The method of claim 11, further comprising habituating a subject or otherwise modulating a desired tract or pathway.

19. The method of claim 11, further comprising modulating one or more regions in one subject and then modulating one or more different regions in the same or different subject or a different subject.

20. The method of claim 11, wherein the magnetic fields are applied to both collect data used to form an image of the nervous tissue in a subject and to modulate said nervous tissue, without moving the subject.
